(19) United States
(12) Patent Application Publication
(10) Pub. No.: US 2010/0210033 A1
(43) Pub. Date: Aug. 19, 2010

(54) PORTABLE DEVICE FOR DETECTING FOOD ALLERGENS

(76) Inventor: Jordan Scott, Wellesley, MA (US)

Correspondence Address: EDWARDS ANGELL PALMER & DODGE LLP, P.O. BOX 55874, BOSTON, MA, 02205, US

(21) Appl. No.: 12/682,036

(22) PCT Filed: Oct. 7, 2008
(86) PCT No.: PCT/US08/79120

§ 371 Date: Apr. 7, 2010

Related U.S. Application Data
(60) Provisional application No. 60/978,124, filed on Oct. 7, 2007.

(51) Int. Cl. G01N 33/53; G01N 30/00

(52) U.S. Cl. 436/501; 422/69

(57) ABSTRACT

The invention relates to a portable device and method for detecting allergenic substances.

### CROSS-REFERENCE TO RELATED APPLICATION

This application claims the benefit of U.S. provisional patent application No. 60/978,124, filed Oct. 7, 2007, the contents of which are incorporated herein by reference.

### BACKGROUND OF THE INVENTION

Many Americans suffer from allergies to substances commonly found in food. It has been estimated that in the United States, up to 2 percent of adults and up to 8 percent of children, particularly those under three years of age, suffer from food allergies (about 5 million people), and this prevalence is believed to be increasing. Common food allergens include shellfish, peanuts, tree nuts, fish, dairy products, wheat gluten and eggs. Allergic reactions can result in serious illness or death, and treatment for an acute allergic reaction, while generally effective, must be administered promptly to avoid injury.

Although individuals who suffer from allergies can try to avoid known sources of allergens, it can be difficult to avoid exposure to allergenic substances altogether. For example, an individual who suffers from peanut allergy would attempt to avoid foods containing peanuts and peanut products; however, in prepared food products, it may be difficult to determine whether an allergenic ingredient is present. In addition, food products can be accidentally contaminated with allergenic substances. In restaurants and other settings where food ingredients may not be known, an allergy sufferer may be exposed to an allergen despite efforts to avoid such exposure.

### SUMMARY OF THE INVENTION

The invention relates to a portable device and system for detecting a potential allergen in a sample, and to methods for detecting the presence or absence of allergens in the sample using a portable system.

In one aspect, the invention provides a portable device for detecting a potential allergen in a sample. The portable allergen detection device can include a housing for receiving an allergen detection chip; a sample inlet port; and an indicator that indicates the presence of the potential allergen in the sample.

In one aspect, the invention provides a portable system for detecting a potential allergen in a sample. The system can include a portable allergen detection device comprising: a housing for receiving an allergen detection chip; a sample inlet port; and an indicator that indicates the presence of the potential allergen in the sample. In certain embodiments, the system includes an allergen detection chip comprising a reagent for detecting the potential allergen (such as an antibody to the potential allergen), the reagent (e.g., the antibody) being labeled with a detectable tag.

In another aspect, the invention provides a portable system for detecting a potential allergen in a sample. The system can include a portable allergen detection device comprising: a housing for receiving an allergen detection chip; a sample inlet port; an indicator that indicates the presence of the potential allergen in the sample; and an allergen detection chip comprising an antibody to the potential allergen, the antibody being labeled with a detectable tag.

In certain embodiments, the indicator for indicating the presence of the potential allergen in the sample comprises a window in the housing for viewing a test result displayed on the allergen detection strip or chip. In certain embodiments, the indicator for indicating the presence of the potential allergen in the sample comprises a display readout for a test result. In certain embodiments, the display readout is a digital display.

In certain embodiments, the system further includes: a detector disposed in the housing that detects the presence or absence of the potential allergen; a microprocessor disposed in the housing for determining the presence or absence of the potential allergen detected by the detector and displaying a result on the digital display; and a power source disposed in the housing for powering the detector, the microprocessor, and the digital display.

In certain embodiments, the sample inlet port is in fluid communication with the allergen detection chip when the allergen detection chip is inserted in the housing.

In certain embodiments, the allergen detection chip comprises an absorbent or bibulous material defining a sample receiving region and an allergen capture region.

In certain embodiments, the absorbent or bibulous material further comprises the reagent (e.g., a labeled antibody to the potential allergen).

In certain embodiments, the allergen capture region further comprises a capture antibody for binding to a potential allergen in the sample.

In certain embodiments, the allergen detection chip comprises a plurality of antibodies to a plurality of potential allergens, each of the plurality of antibodies being labeled with a detectable tag. The plurality of antibodies may be located in the same or in different regions of the allergen detection chip.

In certain embodiments, the potential allergen is a proteinaceous food allergen, which may be a peanut allergen.

In certain embodiments, the sample is a food sample.

In another aspect, the invention provides a method for detecting a potential allergen in a sample. The method includes the step of testing the sample for the allergen with a portable system comprising: a portable allergen detection device comprising: a housing for receiving an allergen detection chip; a sample inlet port; an indicator for indicating the presence of the potential allergen in the sample; and an allergen detection chip comprising a reagent for detecting the potential allergen (such as an antibody to the potential allergen), the reagent (e.g., the antibody) being labeled with a detectable tag.

In another embodiment, the invention provides a method for detecting a potential allergen in a sample. The method includes testing the sample for the allergen with a portable system comprising: a portable allergen detection device comprising: a housing for receiving an allergen detection chip; a sample inlet port; and an indicator that indicates the presence or absence of the potential allergen in the sample; and an allergen detection chip comprising an antibody to the potential allergen, the antibody being labeled with a detectable tag. The method includes the steps of: (a) providing a sample to the sample inlet port; (b) detecting the presence or absence of the potential allergen in the sample; (c) displaying an indication of the presence or absence of the potential allergen in the sample at the indicator.

Other features and embodiments of the invention will be apparent from the description that follows.

### BRIEF DESCRIPTION OF THE DRAWINGS

FIG. 1 is a view of a device according to the invention.

### DETAILED DESCRIPTION OF THE INVENTION

The invention relates to systems and methods for detecting allergenic substances, for example, detecting the presence of allergens in food.

In one aspect, the invention provides a portable system for detecting a potential allergen in a sample. The system includes a portable allergen detection device comprising: a housing for receiving an allergen detection chip; a sample inlet port; and an indicator that indicates the presence or absence of the potential allergen in the sample; and an allergen detection chip comprising a reagent for detecting the potential allergen (such as an antibody to the potential allergen), the reagent (e.g., the antibody) being labeled with a detectable tag.

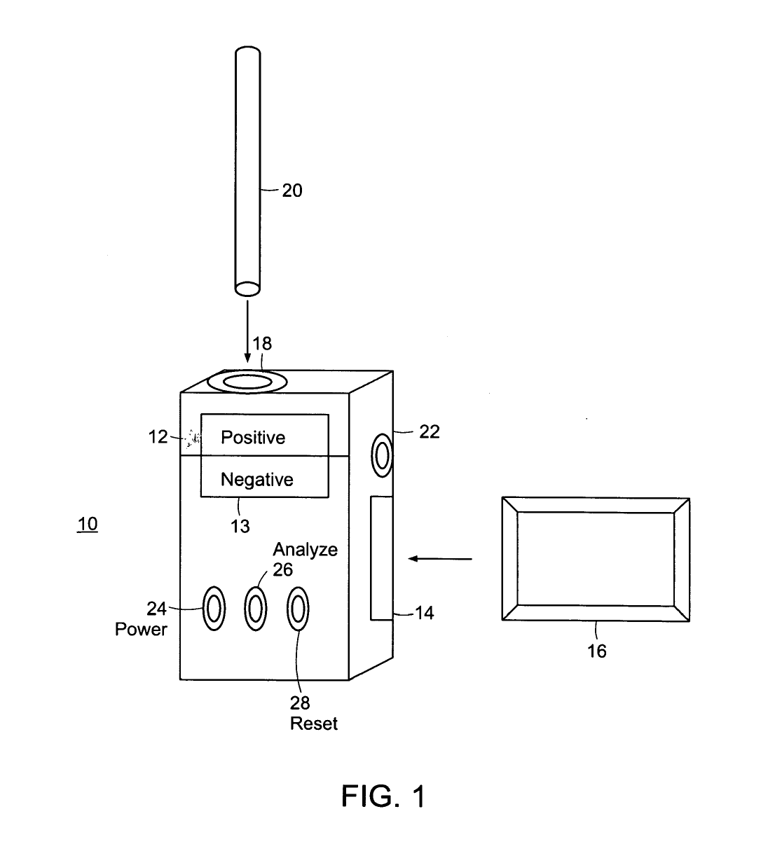

As shown in FIG. 1, a device or system 10 according to the invention can include a housing 12 which has an opening 14 to receive an allergen detection cartridge or chip 16. The housing 12 also includes an opening or sample inlet port 18 for application of a sample (such as a food sample) for testing to the device. The sample can be applied to the inlet port using a sampling device 20, such as a pick, swab, or other probe for providing a sample to the inlet port. The sampling device can be reusable or can be disposable; use of a fresh sampling device for each sample reduces the risk of contamination of samples. Housing 12 can be made of any suitable material, such as metal or plastic, preferably a durable impact-resistant plastic. In certain embodiments, the device is sized for portable handling and use; for example, the housing 12 can have dimensions such that the device will fit into a carrying case or in a pocket for carrying. For example, in certain embodiments, the device including housing 12 can have outer wall dimensions not exceeding 10 cm in width, 15 cm in height, and/or 5 cm in thickness; in certain embodiments, the outer wall dimensions can be no more than 10, 8, 7, 6, 5, 4, 3, or 2 cm in width, no more than 15, 12, 10, 8, 6, 5, 4, 3, or 2 cm in height, and/or no more than 5, 4, 3, 2, or 1 cm in thickness. In certain embodiments, the device weighs not more than 500 gm, preferably not more than 400 gm, still more preferably not more than 300, 200, 100, or 50 gm. The allergen detection chip 16 can be inserted into opening 14 by the user, and after use can be removed manually, or, in certain embodiments, by ejecting the chip with ejection switch 22, which actuates a spring or other means for urging the chip out of the opening 14.

The allergen detection chip 16, which can be reusable or disposable, includes a substrate (e.g., a plastic strip or other member) and one or more reagents capable of reacting with one or more allergenic substances in a sample of interest. The use of reagent-impregnated test strips in specific binding assays, such as immunoassays, is well known. In certain embodiments, a sandwich assay can be employed, in which two reagents (such as antibodies) bind to an analyte (such as a potential allergen) of interest; when the two reagents are bound to the analyte and thereby in close proximity, a detectable signal is generated, indicating the presence of the analyte. An example of such a detection system is a system based on fluorescent resonant energy transfer (FRET), in which one reagent includes a donor fluorophore, and a second reagent includes an acceptor fluorophore. In FRET, light energy is added at the excitation frequency for the donor fluorophore, which transfers some of this energy to the acceptor, which then re-emits the light at its own emission wavelength.

In another embodiment, the allergen detection chip 16 includes a porous or bibulous test strip impregnated with a labeled reagent. In this embodiment, a fluid sample is applied to one portion of the test strip and is allowed to permeate through the strip material. As the fluid permeates the strip, the sample moves into or through a detection zone in the test strip wherein a specific binding reagent is immobilized. Analyte present in the sample can participate in a sandwich or a competition reaction within the detection zone, with a labeled reagent which can also be incorporated in or on the test strip or applied thereto. The immobilized reagent in the detection zone is preferably a highly specific antibody, and more preferably a monoclonal antibody. In certain embodiments of the invention involving a sandwich reaction, a labeled reagent is also preferably a highly specific antibody, and more preferably a monoclonal antibody. Examples of devices using this general approach are found in patent publications such as GB 1589234, EP 0225054, EP 0183442, and EP 0186799. If desired, an absorbent “sink” can be provided at the distal end of the strip material to ensure that sample fluid flow continues through the detection zone.

It will be appreciated that the allergen detection chip may have multiple reagents for the detection of multiple potential allergens (e.g., 1, 2, 3, 4, 5, 10, 20, or more allergens). In certain embodiments, the potential allergen or allergens are food allergens. In certain embodiments, the food allergen is selected from an allergenic protein or protein fragment from a foodstuff, such as shellfish, peanuts, tree nuts, dairy, fish or eggs. In certain embodiments, the reagent is an antibody, which, in certain embodiments, is labeled with a detectable label. Examples of detectable labels include colored latex particles, gold sols, non-metallic colloids, fluorescent dyes, and the like. In preferred embodiments, a detectable label can be used to produce an analytical result without the need to add further reagents in order to develop a detectable signal. In certain embodiments, the label is a latex particle, such as a colored latex particle which can be readily visible to the eye if it becomes bound in the detection zone. If desired, the assay result can be read instrumentally, e.g. by color reflectance. Alternatively, the label can include a fluorophore which can respond to applied electromagnetic energy such as ultraviolet light or visible light, to provide an emitted signal that can be measured instrumentally (e.g., qualitatively or quantitatively).

The device 10 also includes an indicator that indicates, qualitatively or quantitatively, the presence of the potential allergen in the sample. The indicator can be, e.g., a window in the housing through which a user can view a test result on the displayed on the allergen detection chip. For example, if a visually detectable label is used on a labeled reagent in the allergen detection chip, then the user can view the presence or absence of a visually apparent indicator to determine whether a suspected allergen is present in the sample. Alternatively, the indicator can be a device for detecting the presence or absence of a detectable label and displaying the result to a user. The device can include a display, such as a liquid crystal digital display. Alternatively, the indicator can be a lighted indicator or indicators, which may optionally provide an indication by color of the result (e.g., a green light indicting the absence of the selected potential allergen, or a red light indicating the presence of an allergen). As shown in FIG. 1, the display 13 may take the form of indicators for positive and negative results for the presence of a test allergen.

Detectable labels can be detected using any technique known in the art. For example, with a labeled reagent having a fluorescent label, a light source for irradiating a sample with an irradiating wavelength of light can be used, together with a fluorescence detector for detecting at least one emitted wavelength of fluorescent light generated by the label in response to the irradiating wavelength of light. The fluorescence signal can be measured and displayed on a display screen or other indicator to provide a result to the user.

The device 10 may include conventional components such as a central processing unit (CPU) or microprocessor, computer memory connected to the CPU, a timer, a detector (e.g., as described above) and a power source such as a battery in electrical connection with the CPU (not shown). The device can be turned on or off using the power switch or button 24 and can be reset by the user with reset or interrupt button 28. Operation of an analysis can begin automatically, e.g., by use of an actuator or sensor located in the housing proximate the inlet port 18; when the actuator or sensor is triggered by the insertion of a sample probe into the inlet port, the CPU begins the analysis. Alternatively, the device can include a button or switch 26 for manually beginning the analysis.

In another aspect, the invention provides a method for detecting a potential allergen in a sample. The method includes the step of testing the sample for the allergen with a portable system including a portable allergen detection device comprising: a housing for receiving an allergen detection chip; a sample inlet port; and an indicator that indicates the presence of the potential allergen in the sample; and an allergen detection chip comprising a reagent for detecting the potential allergen (such as an antibody to the potential allergen), the reagent (e.g., the antibody) being labeled with a detectable tag. The sample inlet port is in fluid communication with the allergen detection chip when the chip is inserted into the housing.

In another aspect, the invention provides a method for detecting a potential allergen in a sample by testing the sample for the allergen with a portable system. The portable system includes a portable allergen detection device including: a housing for receiving an allergen detection chip, a sample inlet port, and an indicator that indicates the presence or absence of the potential allergen in the sample; and an allergen detection chip comprising an antibody to the potential allergen, the antibody being labeled with a detectable tag. The sample inlet port is in fluid communication with the allergen detection chip when the chip is inserted into the housing. The method includes the steps of: (a) providing a sample to the sample inlet port; (b) detecting the presence or absence of the potential allergen in the sample; (c) displaying an indication of the presence or absence of the potential allergen in the sample at the indicator. The device may also include a detector and/or a readout for displaying a result.

In an exemplary operation, a user obtains a sample of a test substance, such as a food product, e.g., by dipping or touching a food sample with a sampling device. Additional liquid such as water may be added to the sample if needed to provide sufficient fluid for the assay. A suitable allergen detection chip is selected to detect the potential allergen or allergens of interest. The food sample is then applied to the allergen detection chip through the inlet port. The detectable reagent or reagents in the allergen detection chip then bind to the potential allergen (if present) and the result of the assay is displayed at the indicator that indicates the presence of the potential allergen in the sample.

The device is preferably sized and configured for use by an individual (e.g., a patient) who needs to detect food allergens, e.g., to avoid accidental ingestion of allergenic substances. The device can be packaged or labeled with instructions for use, e.g., for the detection of allergens in food.

Without further description, it is believed that one of ordinary skill in the art can, using the preceding description and the illustrative examples, make and utilize the systems and devices of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A portable system for detecting a potential allergen in a sample, the system comprising:
a portable allergen detection device comprising: a housing for receiving an allergen detection chip; a sample inlet port; and means for indicating the presence of the potential allergen in the sample; and
an allergen detection chip comprising an antibody to the potential allergen, the antibody being labeled with a detectable tag.

2. The portable system of claim 1, wherein the means for indicating the presence of the potential allergen in the sample comprises a window in the housing for viewing a test result displayed on the allergen detection chip.

3. The portable system of claim 1, wherein the means for indicating the presence of the potential allergen in the sample comprises a display readout for a test result.

4. The portable system of claim 3, wherein the display readout is a digital display.

5. The portable system of claim 4, further comprising: detection means for detecting the presence or absence of the potential allergen; a microprocessor disposed in the housing for reading determining the presence or absence of the potential allergen detected by the detection means and displaying a result on the digital display; and a power source for powering the detection means, the microprocessor, and the digital display.

6. The portable system of claim 1, wherein the sample inlet port is in fluid communication with the allergen detection chip when the allergen detection chip is inserted in the housing.

7. The portable system of claim 1, wherein the allergen detection chip comprises a bibulous material defining a sample receiving region and an allergen capture region.

8. The portable system of claim 7, wherein the bibulous material further comprises the labeled antibody to the potential allergen.

9. The portable system of claim 8, wherein the allergen capture region further comprises a capture antibody for binding to a potential allergen in the sample.

10. The portable system of claim 1, wherein the allergen detection chip comprising a plurality of antibodies to a plurality of potential allergens, each of the plurality of antibodies being labeled with a detectable tag.

11. The portable system of claim 1, wherein the potential allergen is a proteinaceous food allergen.

12. The portable system of claim 12, wherein the proteinaceous food allergen is a peanut allergen.

13. The portable system of claim 1, wherein the sample is a food sample.

14. A method for detecting a potential allergen in a sample, the method comprising testing the sample for the allergen with a portable system comprising:
a portable allergen detection device comprising: a housing for receiving an allergen detection chip; a sample inlet port; and means for indicating the presence of the potential allergen in the sample; and
an allergen detection chip comprising an antibody to the potential allergen, the antibody being labeled with a detectable tag.

15. A method for detecting a potential allergen in a sample, the method comprising testing the sample for the allergen with a portable system comprising:
a portable allergen detection device comprising: a housing for receiving an allergen detection chip; a sample inlet port; and an indicator that indicates the presence or absence of the potential allergen in the sample; and
an allergen detection chip comprising an antibody to the potential allergen, the antibody being labeled with a detectable tag;
the method comprising the steps of:
(a) providing a sample to the sample inlet port;
(b) detecting the presence or absence of the potential allergen in the sample;
(c) displaying an indication of the presence or absence of the potential allergen in the sample at the indicator.

16. A portable device for detecting a potential allergen in a sample, the device comprising: a housing for receiving an allergen detection chip; a sample inlet port; and an indicator that indicates the presence of the potential allergen in the sample.
